(19) United States
(12) Patent Application Publication
(10) Pub. No.: US 2022/0257550 A1
(43) Pub. Date: Aug. 18, 2022

(54) REDUCING THE VIRAL ACTIVITY OF ELAFIBRANOR WITH RIBOFLAVIN OR DHA

(71) Applicant: DSM IP ASSETS B.V., Heerlen (NL)

(72) Inventors: Igor BENDIK, Kaiseraugst (CH); Hubert Paul HUG, Kaiseraugst (CH); Bernd MUSSLER, Kaiseraugst (CH); Ana TSKEVDIANI, Kaiseraugst (CH)

(21) Appl. No.: 17/625,295

(22) PCT Filed: Jun. 16, 2020
(86) PCT No.: PCT/EP2020/066652

§ 371 Date: Jan. 6, 2022

(30) Foreign Application Priority Data
Jul. 9, 2019 (EP) 19185198.9

(51) Int. Cl. A61K 31/202; A61K 31/192; A61K 31/525; A61P 31/12

(52) U.S. Cl. CPC A61K 31/202; A61P 31/12; A61K 31/525; A61K 31/192

(57) ABSTRACT

The present invention relates to a combination of elafibranor with either riboflavin or DHA as antiviral agent. The bacteriophage T4 titer on E. coli that is increased by elafibranor in comparison to an untreated control is reversed by the combination of elafibranor with riboflavin or elafibranor with DHA. Therefore, these combinations decrease adverse side effects of elafibranor on the gut microbiota.

### BRIEF DESCRIPTION OF THE INVENTION

The present invention relates to the decrease of the elafibranor-induced bacteriophage T4 titer with riboflavin or docosahexaenoic acid (DHA). Effects of the combination of elafibranor with riboflavin or elafibranor with DHA are detected for the first time.

### BACKGROUND OF THE INVENTION

Viruses are particles consisting of nucleic acids and proteins which need living cells for replication and propagation. Bacteriophages or simply phages are viruses that infect bacteria. They are very specific and cannot infect human or other eukaryotic cells. During the lytic cycle the bacteriophage infects a bacterial cell; then it uses the host cell's replication and translation machinery to replicate and lyse resulting in the release of new phages into the environment.

Phage T4 is obligate lytic and Escherichia coli (E. coli) specific. Furthermore, it has a narrow host range on E. coli (Cieplak et al 2018. Gut Microbes. 9(5): 391-399.

Phage T4 is an intestinal resident of humans and can be administered orally without side effects at a dose of up to 105 PFU/ml (Bruttin et al 2005 Antimicrob Agents Chemother. 49(7):2874-8).

Elafibranor is a dual PPARα/δ agonist (Ratziu et al., Gastroenterology 150, 1147-1159, 2016) whose structure is shown below.

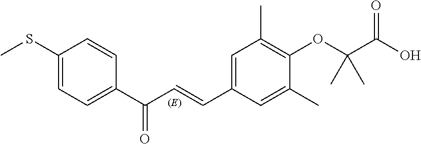 [SMILES: CSc1ccc(C(=O)/C=C/c2cc(C)c(OC(C)(C)C(=O)O)c(C)c2)cc1]

It is currently evaluated in a phase III human trial for possible treatment of nonalcoholic steatohepatitis (NASH) (RESOLVE-IT, https://clinicaltrials.gov/ct2/show/NCT02704403). It also was reported to have beneficial effects on serum levels of glucose, triglycerides, and cholesterol. Unexpectedly, elafibranor increased the titers of phage T4 in an E. coli infection assay. It would be desirable to have a method of reducing or eliminating this side effect of elafibranor.

### DETAILED DESCRIPTION OF THE INVENTION

It has been found that both riboflavin and DHA have anti-phage activities and thus can be used to prevent or control phage infections, such as those observed with elafibranor. Further, we have demonstrated that when the pharmaceutical elafibranor was given together with either riboflavin or DHA, elafibranor no longer increases the phage T4 titers. Further, the expected adverse side effects of elafibranor on the gut microbiota, i. e. the increase of phage titers in the large intestine, is ameliorated by riboflavin and/or DHA.

Thus one embodiment of this invention is a method of treating, preventing and/or lessening the effect of a bacteriophage infection by administering an anti-phage effective amount of riboflavin and/or DHA to an animal, including a human, in need thereof. In some embodiments the phage which is targeted is T4.

Another embodiment of this invention is a method of treating, preventing and/or lessening the effect of a bacteriophage infection in an animal, including a human, associated with the administration of a compound according to Formula I:

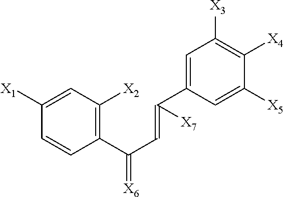

in which:

X₁ is a halogen, R1 or -G1-R1;

X₂ is hydrogen, hydroxy or an unsubstituted alkyloxy;

X₃ is -R3 or -G3-R3;

X₄ is a -R4 or -G4-R4;

X₅ is -R5 or -G5-R5;

X₆ is oxygen;

R1, R3 and R5, which are the same or different, are an unsubstituted alkyl having from one to seven carbon atoms;

R₄ is an alkyl having from one to seven carbon atoms substituted by a group 1 substituent;

G1, G3, G4, and G5, which are the same or different, are oxygen or sulphur wherein at least one of X₁,

X_{3,} X₄ and X₅ is G1R1, G3R3, G4R4 and G5R5, respectively, said group I substituent being selected from the group consisting of -COOR₆ and -CONR₆R_{7,} wherein R₆ and R₇, which are the same or different, are hydrogen or an unsubstituted alkyl having from one to seven carbon atoms, or an optical isomer, a geometric isomer, a racemate, a tautomer, a salt or mixtures thereof.

One embodiment of this invention is a composition comprising a compound of Formula 1 and riboflavin and/or DHA. Another embodiment is the use of DHA or riboflavin to reduce the T4 phage activity observed in an animal including a human, which may or may not be associated with the administration of compounds of Formula 1.

In preferred embodiments the compound of Formula I is elafibranor, or a salt thereof.

A further embodiment of this invention is a method of decreasing bacteriophage titers associated with elafibranor therapy comprising administering riboflavin and/or DHA to a person who is undergoing elafibranor therapy. In some embodiments the animal, including a human, undergoing such treatment has symptoms of non-alcoholic fatty liver disease, (NALFD), non-alcoholic steatohepatitis (NASH), hyperglycemia, high cholesterol levels, and/ or high triglyceride serum levels.

A further embodiment of this invention is the use of riboflavin to decrease the T4 titer associated with elafibranor therapy. Yet another embodiment of this invention is the use of DHA to decrease the T4 titer associated with elafibranor therapy. A further embodiment of this invention is the use of riboflavin and DHA to decrease the T4 titer associated with elafibranor therapy.

In yet another embodiment of this invention the animal, including a human, is not exhibiting symptoms of a disease which requires elafibranor therapy, but is exhibiting symptoms of an elevated

T4 titer in the gastrointestinal tract. These symptoms can include intestinal discomfort, diarrhea, cramping, and can be attributed to the disruption of the natural population of microbacterial flora in the gut.

### BRIEF DESCRIPTION OF THE FIGURES

FIG. 1: Phage T4 and host bacteria E.coli 613 were mixed at an MOI of 0.01, and incubated for 3 hours at 30° C. without compounds (control), or elafibranor (DSM 1, 0.02 mg/ml), riboflavin (DSM5, 0.02 mg/ml), DHA (DSM 8, 0.02 mg/ml) or elafibranor together with either riboflavin (DSM 15, 0.02 mg/ml each) or DHA (DSM 18, 0.02 mg/ml each). The number of phage particles (PFU) is given. Experiment were done in triplicate.

FIG. 2: Phage T4 and host bacteria E.coli 613 were mixed at an MOI of 1000/1, and incubated for 3 hours at 30° C. without compounds (control), or elafibranor (DSM 1, 0.02 mg/ml), DHA (DSM 8, 0.02 mg/ml), or elafibranor together with DHA (DSM 18, 0.02 mg/ml each). The number of phage particles (PFU) is given. Experiment were done in triplicate.

### DEFINITIONS

Elafibranor is a compound of Formula 1 which is shown below:

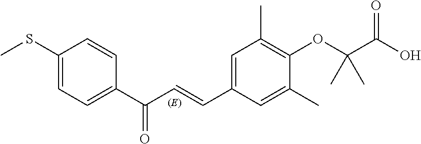 [SMILES: CSc1ccc(C(=O)/C=C/c2cc(C)c(OC(C)(C)C(=O)O)c(C)c2)cc1]

“Bacteriophage T4” means a double-stranded DNA bacteriophage that infects E. coli.

“Riboflavin” includes the various forms of Vitamin B2, including ribo-5-phosphate. “DHA” includes the various forms of DHA, including ethyl esters. “Co-administering” means that the compound of Formula 1 and the riboflavin and/or DHA are administered either simultaneously or within 4 hours of each other. In preferred embodiments, they are administered simultaneously, either in a same dosage form or in separate dosage forms.

### DOSAGES

A recommended daily dose is the maximal dose of riboflavin or DHA that is allowed by regulatory authorities. The dosages which are effective in exhibiting an anti-T4 bacteriophage effect may vary.

For DHA, (based on the amount of pure DHA in a formulation), a human dose would be at least 500 mg per day, preferably at least one gram per day, and more preferably 2 to 3 grams per day, although more may be administered. DHA does not have an upper limit due to toxicity considerations, so amounts in excess of 2 grams per day may be administered without safety concerns. For animals, the dosages can be adjusted according to the animal's weight, based on the human considerations where a human is considered to have a weight of 70 kilograms. In another embodiment, the dosages for either the animal or human is a supra-physiological dose, which is a dose above that which is the daily required amount of DHA

For riboflavin, the nutritional requirement for a person is 1.6 mg per day, so the recommended dosage according to this invention would be from 1-100 mg per day in excess of that normally consumed in a balanced diet 1.6 mg per day). For a non-human animal, the amount according to this invention would be from 1-100 mg above the animals' normal daily requirement. The upper limit on the amount of riboflavin consumed before it is toxic is very high, so these amounts are within the human or animal's safety limit/

In another embodiment, a supra-physiological dose of both DHA and riboflavin is administered, which is an amount which is above the normal daily requirement of the animal.

Elfibranor is administered in dosages which are known in the art for the particular condition being treated. In one embodiment, for use in combination with elafibranor, the formulation comprises both elfibranor and riboflavin and/or DHA. In another embodiment, the animal, including the human receiving elafibranor is administered a dosage of elafibranor and a separate dosage of riboflacin and/or DHA. The separate dosage may be administered simultaneously or it may be administered at a different time throughout the day.

### FORMULATIONS

The composition of the present invention is preferably in the form of nutritional composition, such as fortified food, fortified feed, or fortified beverages, or in form of fortified liquid food/feed (such as drinks, or shots), pills or capsules for animals including humans.

Non-human animals including companion animals (such as dogs, cats, and horses) and animals reared for their milk production (such as dairy cows, buffalo, sheep and goats) may also exhibit symptoms of

NALFD and/or NASH. Further, these animals may also exhibit gastric symptoms related to a high T4 activity affecting their normal microbial flora.

The dietary and pharmaceutical compositions according to the present invention may be in any galenic form that is suitable for administering to the animal body including the human body, especially in any form that is conventional for oral administration, e.g. in solid form, such as (additives/supplements for) food or feed, food or feed premix, fortified food or feed, tablets, pills, granules, dragées, capsules, and effervescent formulations such as powders and tablets, or in liquid form such as solutions, emulsions or suspensions as e.g. beverages, pastes and oily suspensions. The pastes may be encapsulated in hard or soft-shell capsules, whereby the capsules feature e.g. a matrix of (fish, swine, poultry, cow) gelatin, plant proteins or lignin sulfonate. Examples for other application forms are forms for transdermal, parenteral or injectable administration. The dietary and pharmaceutical compositions may be in the form of controlled (delayed) release formulations.

### EXAMPLE 1

### The Anti-Phage Activity of Elafibranor is Reversed By Either Riboflavin or DHA

Methods:

Elafibranor (GFT505, CAS no. 923978-27-2) was from BioVision, Inc. (San Francisco, Calif., USA). Riboflavin (CAS no. 83-88-5) and Docosahexaenoic acid (DHA, CAS no. 6217-54-5) were from DSM

Nutritional Products Ltd (Kaiseraugst, Switzerland). All stock solutions were in 20 mg/ml DMSO. All compounds were used at a final concentration of 0.02 mg/ml.

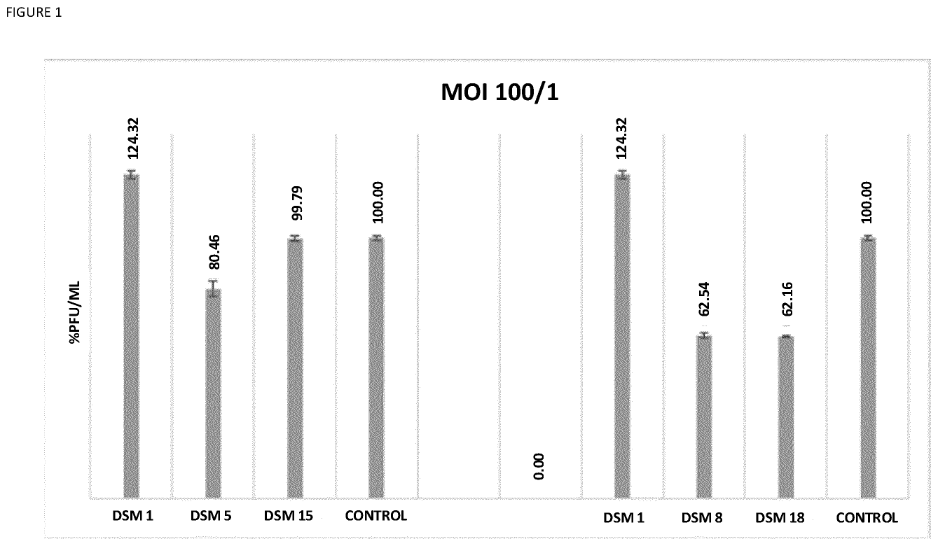

E. coli was grown in liquid LB (lysogeny broth) medium. Phage T4 and host bacteria E. coli were mixed at a multiplicity of infection (the number of viral particles per infected cell, MOI) of 0.01 and incubated for 3 hours at 30° C. with or without compounds as indicated in FIG. 1. The number of phage particles (PFU) was determined by plating on agar.

Results:

The effect on phage T4 titers of elafibranor alone or in combination with either riboflavin or DHA was tested in liquid culture of E. coli with a MOI of 0.01 followed by a plaque assay on agar plates to count the PFU (FIG. 1). Under these conditions, elafibranor significantly increased the PFU of phage T4. Riboflavin or DHA decreased phage T4 PFU compared to untreated controls. In the combination of elafibranor with riboflavin phage T4 PFU was brought back to control levels. In the combination of elafibranor with DHA phage T4 PFU was even brought down to the level shown by DHA alone (FIG. 1).

The effect on phage T4 titers of elafibranor alone or in combination with either riboflavin or DHA was tested in liquid culture of E. coli with a MOI of 100/1 followed by a plaque assay on agar plates to count the PFU (FIG. 1). Under these conditions, elafibranor significantly increased the PFU of phage T4. Riboflavin or DHA decreased phage T4 PFU compared to untreated controls. In the combination of elafibranor with riboflavin phage T4 PFU was brought back to control levels. In the combination of elafibranor with DHA phage T4 PFU was even brought down to the level shown by DHA alone (FIG. 1).

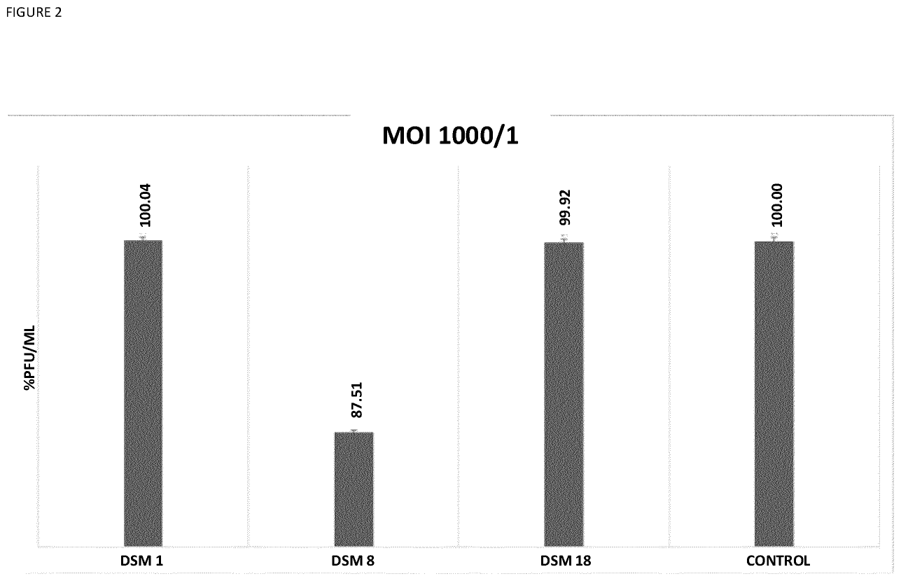

At an MOI of 1000/1 the combination of elafibranor with DHA brings the phage titer back to the control level (FIG. 2).

In summary, riboflavin or DHA counteract the effects of elafibranor on phage T4 titers whereby MOI is a crucial parameter.

Thus, riboflavin as well as DHA can contribute to maintenance of healthy gut microflora regarding bacteriophage titers in patients treated with elafibranor.

1. A method of treating, preventing or lessening a bacteriophage infection in an animal, including a human in need thereof or at risk thereof, comprising administering to the animal an effective amount of riboflavin and/or DHA.

2. A method according to claim 1 wherein the bacteriophage infection is associated with the administration of a compound according to Formula I: 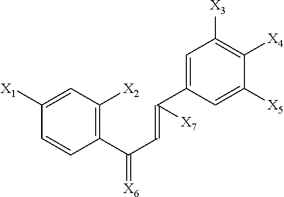
in which:
X₁ is a halogen, R1 or -G1-R1;
X₂ is hydrogen, hydroxy or an unsubstituted alkyloxy;
X₃ is -R3 or -G3-R3;
X₄ is a -R4 or -G4-R4;
X₅ is -R5 or -G5-R5;
X₆ is oxygen;
R1, R3 and R5, which are the same or different, are an unsubstituted alkyl having from one to seven carbon atoms;
R₄ is an alkyl having from one to seven carbon atoms substituted by a group 1 substituent;
G1, G3, G4, and G5, which are the same or different, are oxygen or sulphur wherein at least one of X₁, X₃, X₄ and X5 is G1R1, G3R3, G4R4 and G5R5, respectively, said group I substituent being selected from the group consisting of -COOR₆ and -CONR₆R₇, wherein R₆ and R₇, which are the same or different, are hydrogen or an unsubstituted alkyl having from one to seven carbon atoms, or an optical isomer, a geometric isomer, a racemate, a tautomer, a salt or mixtures thereof comprising:
co-administering to the animal including a human, an anti-phage effective amount of DHA or riboflavin.

3. A method according to claim 2 where the compound of Formula 1 is elafibranor.

4. Use of DHA and/or riboflavin to of treating, preventing or lessening a bacteriophage infection in an animal, including a human.

5. Use according to claim 4 wherein the infection is associated with the administration of a compound according to Formula I: 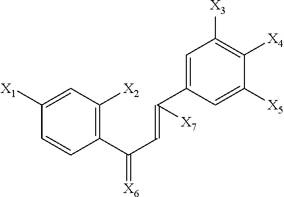
in which:
X₁ is a halogen, R1 or -G1-R1;
X₂ is hydrogen, hydroxy or an unsubstituted alkyloxy;
X3 is -R3 or -G3-R3;
X4 is a -R4 or -G4-R4;
X5 is -R5 or -G5-R5;
X6 is oxygen;
R1, R3 and R5, which are the same or different, are an unsubstituted alkyl having from one to seven carbon atoms;
R4 is an alkyl having from one to seven carbon atoms substituted by a group 1 substituent;
G1, G3, G4, and G5, which are the same or different, are oxygen or sulphur wherein at least one of X₁, X3, X4 and X5 is G1R1, G3R3, G4R4 and G5R5, respectively, said group I substituent being selected from the group consisting of -COOR₆ and -CONR₆R₇, wherein R₆ and R_{7,} which are the same or different, are hydrogen or an unsubstituted alkyl having from one to seven carbon atoms, or an optical isomer, a geometric isomer, a racemate, a tautomer, a salt or mixtures thereof.

6. Use according to claim 5 where the compound is elafibranor.

7. A composition comprising a compound according to Formula 1 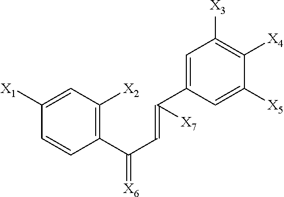
in [symbol]
X₁ is a halogen, R1 or -G1-R1;
X₂ is hydrogen, hydroxy or an unsubstituted alkyloxy;
X₃ is -R3 or -G3-R3;
X₄ is a -R4 or -G4-R4;
X₅ is -R5 or -G5-R5;
X₆ is oxygen;
R1, R3 and R5, which are the same or different, are an unsubstituted alkyl having from one to seven carbon atoms;
R₄ is an alkyl having from one to seven carbon atoms substituted by a group 1 substituent;
G1, G3, G4, and G5, which are the same or different, are oxygen or sulphur wherein at least one of X₁, X₃, X₄ and X5 is G1R1, G3R3, G4R4 and G5R5, respectively, said group I substituent being selected from the group consisting of -COOR₆ and -CONR₆R₇, wherein R₆ and R₇, which are the same or different, are hydrogen or an unsubstituted alkyl having from one to seven carbon atoms, or an optical isomer, a geometric isomer, a racemate, a tautomer, a salt or mixtures thereof; and
riboflavin or DHA.

8. A compound according to claim 7 wherein the compound of Formula 1 is elafibranor.
